(19) United States
(12) Patent Application Publication
(10) Pub. No.: US 2024/0415727 A1
(43) Pub. Date: Dec. 19, 2024

(54) A CONTROL UNIT, SYSTEM AND METHOD FOR ANALYZING USAGE OF COMPRESSION THERAPY

(71) Applicant: Arjo IP Holding Aktiebolag, Malmö (SE)

(72) Inventor: Michael David NEWTON, Boerne, TX (US)

(73) Assignee: Arjo IP Holding Aktiebolag, Malmö (SE)

(21) Appl. No.: 18/699,922

(22) PCT Filed: Oct. 11, 2022
(86) PCT No.: PCT/SE2022/050916

§ 371 Date: Apr. 10, 2024

Related U.S. Application Data
(60) Provisional application No. 63/254,325, filed on Oct. 11, 2021.

(51) Int. Cl. A61H 9/00

(52) U.S. Cl. CPC A61H 9/0078; A61H 2201/5007; A61H 2201/5043; A61H 2201/5056; A61H 2201/5071; A61H 2205/06; A61H 2205/10; A61H 2205/12

(57) ABSTRACT

A control unit, system and method for analyzing usage of compression therapy to a patient's limb or anatomical structure using at least one inflatable compression garment having at least one inflatable chamber.

### TECHNICAL FIELD

The present invention relates to a control unit (i.e. controller), system and method for analyzing usage of compression therapy to a patient's limb or anatomical structure. Especially, the present invention relates to the operation and analysis of a compression therapy system for use with a patient. The compression therapy system including a coupling assembly for fluidically connecting at least one inflatable/deflatable garment to a fluid pressure control system. The present invention further relates to a system and method for analyzing the degree of delivered compression therapy to a patient's limb or anatomical structure over time and identifying and selecting options to improve the delivered prophylaxis.

### BACKGROUND

This invention relates to pneumatic systems and in particular to pneumatic systems having an inflatable/deflatable article, for example a compression garment connected to a fluid source, for example a pump. It is known that the use of such systems often involves multiple individual periods of patient use interspersed with periods of non-use. These periods can occur often in a single day and also spread across multiple nursing shifts and days during a patient's use of the system.

In the light of the above, there is a need for a system and a method, which addresses and mitigates the risk for use error associated with a compression system when the user connects garments and starts the system operation to provide prophylaxis but the patient limbs are not physically fitted within the garment. As a result, the intended prophylaxis is not delivered and this condition may not be discovered for some time. Another associated example is where the system is connected and set up correctly but the garments are subsequently removed by the patient when the clinician in is not present.

### SUMMARY

The present disclosure relates to a system and method for analyzing and promoting the clinical use of compression therapy to a patient's limb or anatomical structure.

The recording of usage data on the operation of a compression system is undertaken. An aspect of some embodiments of the present invention is to provide increased monitoring and indication to the user on the degree of usage with the aim of promoting increased use. It also provides metrics to the clinician based on this monitored usage to help with management of wider aspects of the VTE prophylactic regime for a patient, which seeks to mitigate, alleviate or eliminate one or more of the above-identified deficiencies in the art and disadvantages singly or in any combination.

An aspect of the present invention relates to a system, method and control unit for controlling compression therapy to a patient's limb or anatomical structure. This can involve the application to areas such as foot, calf, thigh or arm individually or in combination.

An aspect of the present invention relates to a control unit for analyzing usage of compression therapy to a patient's limb or anatomical structure by using at least one inflatable compression garment comprising at least one inflatable chamber. The control unit comprising an identification component for detecting a type of at least one compression garment, used during the compression therapy, and located within a connector of the at least one compression garment, an air pump for regulating air pressure to the at least one compression garment based on the detected type, at least one pressure sensor for measuring the pressure present in at least one of the inflatable chambers of the connected at least one garment during the compression therapy and generating signals corresponding to the pressure in the chamber. Further, the pressure sensor is providing signals during the application of compression which signals comprises characteristics of the presence of the limb or anatomical structure within the at least one compression garment. Further, the pressure sensor denoting a change in the compression level of the at least one compression garment as a result of the limb or anatomical structure being removed from the at least one compression garment compared to the characteristic of the signals during the previous compression. Further, a visual indication is provided by the system to the user based on monitored time when the limb or anatomical structure is present in the at least one connected garment and wherein a visual indication is provided by the system to the user based on monitored time when the limb or anatomical structure is detected as not being present in the at least one connected garment.

An aspect of the present invention relates to a compression system for analyzing usage of compression therapy by using at least one compression garment, comprising a user interface. The user interface comprising a first display indicator associated with the usage of the at least one compression garment in a first state of operation of the compression system and a second display indicator associated with the usage of the at least one compression garment in a second state of operation of the compression system.

An aspect of the present invention relates to a method for analyzing usage of compression therapy by using at least one compression garment of a compression system comprising a user interface. The user interface comprising a first display indicator associated with the usage of the compression garment in a first state of operation of the compression system and a second display indicator associated with the usage of the garment in a second state of operation of the compression system. The method comprising monitoring time when the compression garment is in each state of operation.

The features of the above-mentioned embodiments can be combined in any combinations.

### BRIEF DESCRIPTION OF THE DRAWINGS

Further objects, features and advantages of the present invention will appear from the following detailed description of the invention, wherein embodiments of the invention will be described in more detail with reference to the accompanying drawings, in which:

FIG. 1 shows a compression system according to an embodiment of the present invention,

FIG. 2 shows a flowchart of a compression system according to an embodiment of the present invention,

FIG. 3a-d shows an embodiment of a graphical user interface according to an embodiment of the present invention,

FIG. 4 shows a timing diagram of usage of a compression system according to an embodiment of the present invention with the functional aspects of the present invention,

FIG. 5 shows a flowchart of an embodiment of a compression system according to an embodiment of the present invention,

FIG. 6 shows a system diagram of a logical breakdown of an algorithm used to provide usage monitoring functions for a compression system according to an embodiment of the present invention,

FIG. 7 shows a graph visualizing the operation of a usage monitoring function of a compression system according to an embodiment of the present invention, where the usage is greater than a preferred target, and

FIG. 8 shows a graph visualizing the operation of a usage monitoring function of a compression system according to an embodiment of the present invention, showing where the calculated usage is less than a preferred target.

### DETAILED DESCRIPTION

Embodiments of the present invention will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference signs refer to like elements throughout.

Within the field and scope of compression systems intended for Venous Thrombolembolism (VTE) prophylaxis, it is known that these systems are used at different times in the day and for different periods of time. As a result they are used by different staff members during a single day, where responsibility is transferred from shift to shift. As such it is beneficial if the prophylactic or treatment activities of one nursing shift can be seen by a subsequent nursing shift. This is particularly helpful in a busy acute care facility where the overall prophylactic or therapeutic operation of a compression system associated with an individual patient can be readily seen at the compression system by any clinician and at any time.

An aspect of the invention is that the system can utilize its monitoring of prior usage to provide recommendations for changes in operation, or automatically modify settings, or provide a progress indication to the clinician on the degree of compliance with a prescribed target.

A further aspect of the potential use error associated with a compression system is when the user connects garments and starts the system operation to provide prophylaxis but the patient limbs are not physically fitted within the garment. As a result the intended prophylaxis is not delivered and this condition may not be discovered for some time. Another associated example is where the system is connected and set up correctly but the garments are subsequently removed by the patient when the clinician in is not present. The present invention is intended to monitor both these type of examples, records the timing associated with these events and indicate the situation to the clinician through usage monitoring based indications and alarms.

The invention includes and combines two different and separate elements, usage monitoring and performance monitoring.

The usage monitoring is covering the actual usage of the product in a clinical setting. This is based on a combination of the following aspects:
Detection of the type of garment(s) attached to a compression pump of a compression system. This allows us to identify and automatically confirm the type(s) and variants of connected compression garment(s) to the pump and the associated compression monitoring parameters that are optimal for the connected garments, and
Time-based monitoring and recording of the duration of pump operation with the selected garment(s) including the monitoring the recording of the duration of pump operation when the requisite number of limbs is identified as being present in the garments during their inflation. The recording being in the form of a number of data records in the form of a start/stop time elapsed time measurements as well as incrementing elapsed time counters. This provides a plurality of data records with a time-base to allow the system to determine when and for how long and how effectively the prophylaxis is applied or not applied for,

This is combined with the following element
An algorithm-based, multi-parameter analysis of the delivered compression therapy based on data collected over time by the usage monitoring feature. This analysis results in a comparison with a target of prescribed operation that is clinically appropriate for the individual patient.

It is known that simple timers associated with recording elapsed pump use can be included in many products including compression pumps. A novel aspect of the present invention is the analysis of both the individual durations of each delivered patient use of the compression system and the individual durations of non-use of the system.

The analysis is used to calculate a number of metrics associated with the actual degree of historical operation of the compression system.

A further aspect is that at least one calculated metric is provided to the user by means of a LCD screen of the compression system. The form of the metric displayed can be either numeric such as a value, score, percentage or it can be provided in a graphical form such as a bargraph, icon or other non-numerical pictorial form. It is an aspect of the invention that the set of metrics are updated continuously such that a change in the degree of use or non-use of the compression system is reflected in the metrics and the associated visual display. It is an aspect of at least one of the metrics that the relative proportion of recent time in use compared to recent time not in use is compared with the equivalent proportion at an earlier period. This allows for an early identification of any deviation from the long term usage as may occur when either positive or negative effects on product usage occurs.

In addition, one or several of the below listed elements can be incorporated:
Inclusion of a Real Time Clock (RTC) timing device can provide temporal details such as the actual time of day of the system operation. That information can be associated with the daily timing of other aspects of the patient care experience. These care aspects can affect or preclude the use of a VTE system (e.g. bathing/bathroom visits/medical examinations/diagnostic procedures/medication etc.) and their time is normally schedule or otherwise known and hence recorded in the patient care records. Through the display of the metric showing the delivered prophylaxis to date it allows for improvements in management of care, the audit and reporting of VTE prophylaxis for an individual patient to be undertaken.
The RTC also allows for a time-stamped record to be stored within a digital memory in the form of a log of the individual events of and individual events of non-use using a start/stop timestamp-based approach. This includes time and duration of activities associated with the pump being taken out of use by clinician activity such as garment removal from the pump and/or the pump being physically switched off to be clearly recorded. Hence, individual usage episodes are identified and recorded for later analysis and reporting to the clinician as well as inclusion in the patient's electronic medical record (EMR).

An aspect of the invention is to use both start and stop timestamps of events—rather not just recording the elapsed duration of the individual episodes. From this basic data it is possible to build up a detailed and relevant timed log of the use of the system and the delivered prophylaxis. Further records can be included in the log including system alarms and pump operating settings.

Performance Monitoring extends and builds on the previously described usage monitoring data to identify and generate a number of parameters and operational metrics associated with the effectiveness of the delivered prophylaxis to the patient over time. This provides feedback to the user on the amount of delivered prophylaxis over time using an algorithm-based score based on the monitored usage together with other additionally sensed and recorded parameters. For example, the time of day is important as an additional reference and data point when analyzing product usage since human body conditions vary significantly between a patients asleep and awake states and also certain treatments (e.g. pharmaceuticals) are applied at or during certain times that can affect the effectiveness of delivered compression.

The present invention can also utilize a proprietary algorithm to provide visual recommendations to the user if the amount of usage is insufficient, compared to a prescribed or recommended overall threshold or target. This threshold or target can be associated, selected or configured with the identification component associated with each connected garment type or can set more generally by the control system based on clinical input—for example using part of the user interface located on the pump. It is an aspect of the invention that each garment type can set differing thresholds based on an aspect of the identification component located in the connector of the specific garment type attached to the pump through an automatic configuration procedure on connection of the garment to the pump. This allows a garment-specific configuration from a plurality of configurations to be made for the target or threshold. A further aspect of the invention is that this threshold configuration is set at a first value when the garment is newly manufactured and unused and can be modified or adapted to a second value when the same garment is inserted into a compatible approved pump for initial use. This aspect allowing the pump to readily differentiate between a new unused garment on a patient and a previously used garment that may require different operation in terms of compression parameters or monitoring algorithms to be employed.

This same aspect and process is equally applicable when a garment is reprocessed involving cleaning or sterilized after a first patients use and prior to a second patients use, thereby providing a means of indicating that garments that have a limited operational lifetime are identified to the clinician in terms of not being brand new. This aspect combines the threshold setting benefit of the present invention with a secondary benefit associated with the performance monitoring of the garment operation. As compression garments age though us, dependent on the materials used for each type, their material characteristics can change and therefore potentially affect their function or reliability. This result can occur through either the effect of repetitive expansions due to continual use with a single patient, through the effects of multiple reprocessing cleaning and test cycles or though their use with multiple patients. In order to provide optimal operation, the present invention can make use of the identification component to assist an algorithm in measuring the operation of the compression garment when in use.

A further aspect of the invention involves the use of a plurality of identification components (sizes, materials, positions) located in the garment connector such that the pump control system is able to identify each type or family of compression garment uniquely from a plurality of types of compression garments that are being monitored for operation.

The use of an algorithm to provide metrics of usage and sensed compliance during actual use are known in the prior art for intermittent pneumatic compression (IPC) treatment (e.g. Wright U.S. Pat. No. 9,889,063). The present invention differs in that it combines a range of metrics related to VTE prophylaxis, a garment identification component within each of the attached garment connectors and the monitoring of usage of each garment and pump when in operation providing prophylaxis/treatment or when the pump is not in active pneumatic operation, together with making associated recommendations and decisions to aid clinical decision making for both current and future use.

This compression system includes one or more of the following aspects:
The identification of the specific garment type fluidically connected to the pump by means of an identification component located in the garment connector and sensed by the compression system. At least one generated parameter in the form of metric based on the analysis of recent system usage, the parameter being related to the degree of delivered prophylaxis over a defined timeframe for the garment type associated with the identification component.
The at least one parameter having a weighting and forming a part of a decision algorithm performed by software within the controller of the compression system.
The pump automatically switching on or otherwise changing its user interface in response to the connection of the aforementioned garment connector.
The pump automatically starting the application of therapy/prophylaxis based on the connection of the aforementioned garment connector.
The timeframe for analysis being associated or based on true elapsed time (e.g. clock/calendar time such as that monitored by a RTC or equivalent) and associated with at least one of the following; the connection of the aforementioned garment connector, the removal of the aforementioned garment connector from the pump or the use of an element user interface of a pump. The period of time for the analysis being further selectable from a plurality of time periods via the pump control system, sensed identification component or user selection and encompassing various different selectable options such as between specific selectable dates/times or timing durations. Alternatively, the period can more simply involve a duration including the most recent hours or days or over a longer term totalization of an individual patient's use of the system.
A generated parameter in the form of a calculated metric associated with the monitored recent system usage compared to the recent monitored lack of usage of the compression system as defined as when the compression system is intentionally not in operation (such as when switched off or not delivering prophylaxis).
A generated parameter in the form of a calculated metric associated with the monitored usage of the system compared to the recent monitored usage when the compression system is intentionally in use but when garments are sensed as not being fitted to a limb or are removed from a limb.
A generated parameter in the form of a calculated metric associated with the usage of the recent system associated with each available system operating modes or types of prophylaxis. The range of operating modes/prophylaxis types being selectable within the control system.
A generated parameter in the form of a calculated metric associated with recent system usage and the connected garment type(s).
A generated parameter in the form of a metric associated with recent system usage and the alarm status of the system and the associated connected garment(s).
A generated parameter in the form of a metric associated with recent system usage and involving the measurement of sensed parameters associated with the applied prophylaxis (such as measured from the limb).
A generated parameter in the form of at least one calculated metric associated with recent system usage and at least one clinical parameter associated with the patient using the system. The clinical parameter can be measured by the compression system as a separate function to the compression or alternatively provided by an external patient monitoring device. A range of different clinical parameters can be utilized including examples such as vital signs (pulse rate, temperature, respiration rate, blood pressure) as well as other related clinical measurements (for example such as oxygenation saturation level, risk factor and degree of mobility).
A generated parameter in the form of calculated metrics associated with recent system usage and a variable associated with the patient's VTE risk factors, general condition and care plan.
An algorithm, operating within the compression system, using at least one weighted parameter, providing a calculated metric for use by the compression system in its operation and/or communication to the clinician.

The above aspects and parameters can be used to provide a patient-specific aspect to the analysis of system usage. Further, the above aspects and parameters can also be used to provide a compression system-specific aspect to the analysis of compression system usage over its use with multiple patients.

The calculation of the parameters and metrics described above is performed within the compression system using software running on a microcontroller within the compression system. The metrics are calculated on a periodic basis and updated for use by the parameters within the algorithm.

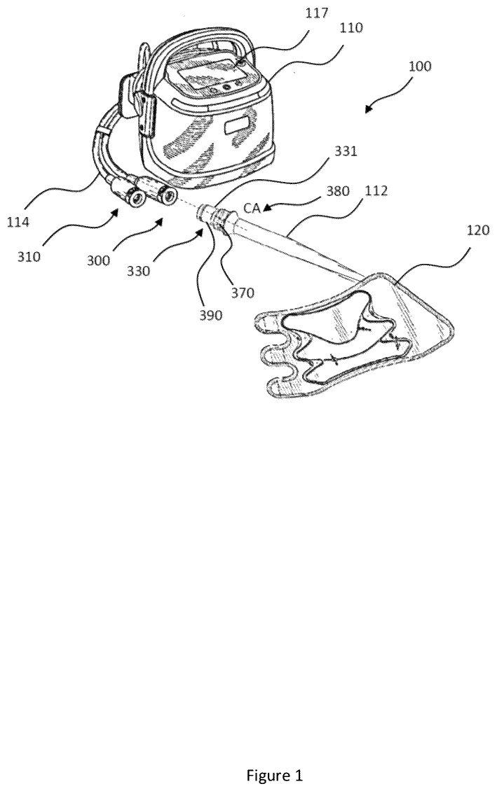

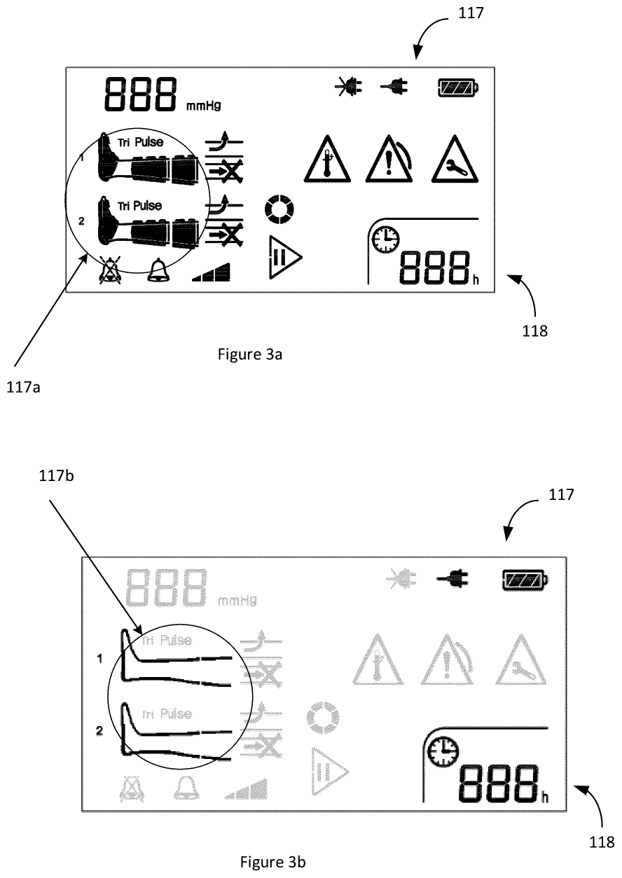

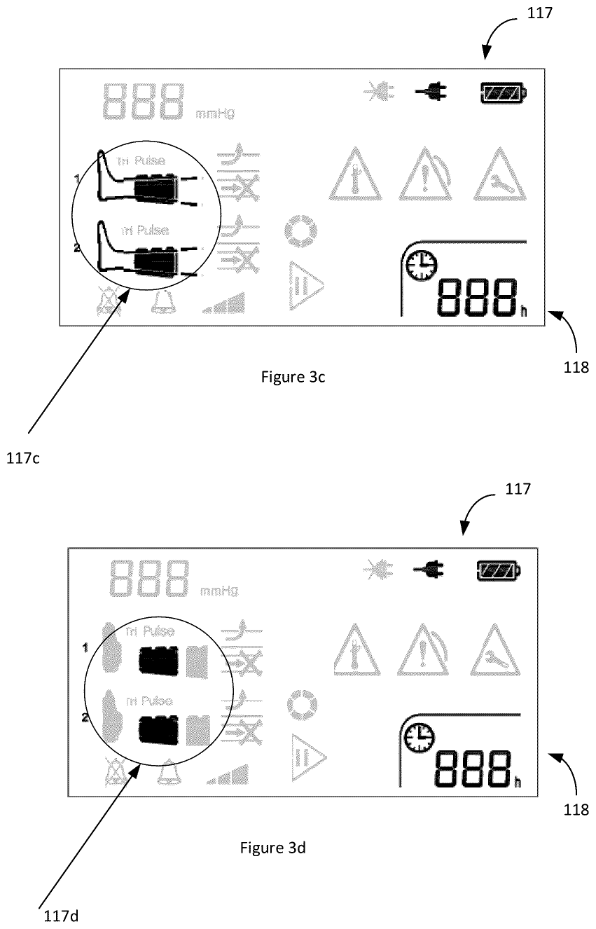

The parameters and metrics are used within the present invention using a proprietary algorithm to provide an estimation of the effectiveness of current system usage together with predictions of future utilization and expected prophylactic performance against a target, threshold or required degree of use. The results of the algorithm can be displayed to the user to recommend a manual change in usage or operation or alternatively be used to automatically initiate a change in system operation with the aim of promoting increased usage and/or effectiveness of the compression system. The algorithm results can be communicated locally via visual indications and/or communicated more widely by the system for recording in individual patient records or other facility management systems. Examples of the visual indications to the user can be via a user interface as shown on the compression system in FIG. 1 and FIGS. 3a-d.

An aspect of the invention is that these monitored and calculated individual usage and effectiveness parameters are stored and can be recalled for further analysis as part of individual patient records, facility management and as part of enhanced system operation.

Each parameter is provided with a weighting so that the relative effect and importance of each different parameter can be considered in the algorithm calculation.

It is within the scope of the invention that these individual parameter weightings can be modified by the user or the algorithm itself in an adaptive manner. The individual parameters and metrics being individually associated with the specific garment type used as indicated by the identification component associated with the connected compression garment.

A further aspect of the invention is that the compression system includes the use of artificial intelligence techniques to update its internal algorithmic operation such as using a machine-based learning approach. This can involve a range of software-based techniques including multi-parameter analysis, predictive analytics, statistical optimizations and heuristics to further enhance the algorithm over prolonged use. The compression system has the ability to be adaptive in nature and refine the algorithm including elements of its operation, format or construction based on the results obtained from multiple patient usage episodes. Hence, the algorithm is able to improve its function and operation based on data gathered from the experience gained in real life system usage. Associated with this machine learning aspect of the operation of the compression system, the algorithm is able to provide additional metrics on its learning status, development and enhancements for subsequent interpretation and assessment.

The compression system is also able to modify its function in terms of its operational compression parameters such as pressure level(s), cycle time, inflation rate, compression duration and other features. This is based on the results of the algorithm, its parameters and metrics and the type of compression garment attached to the system as defined by the identification component(s) located in the connector(s) of the attached garment(s). This approach provides an alternative means of operation and allows for an enhanced compression system operation compared to the fixed and defined operation available in the prior art. This enhancement is made possible through the algorithmic analysis of data associated with the combination of the system monitored usage and the detection of the specific connected garment type(s).

The individual parameters and outputs from the algorithm can be reported to the user (e.g. via the interface) communicated remotely (e.g. via wireless communication) or utilized by the compression system in the selection or modification of the compression system operating parameters.

As a result of this communication capability, another aspect of the invention is that the compression system can automatically modify its current operating mode based on the results of the analysis of its previous use and performance. This allows an automatic change in the delivered prophylaxis based on the degree of use and allows for an adaptive mode of operation, which can result in improvements in both system operation and associated patient care, for example after periods of sub-optimal usage. Aspects of this adaptive change in the prophylaxis operation provided include modifications the typical compression parameters including for example pressure waveforms, garment pressure level, cycle time.

The algorithm can be configured to provide an advisory indication to the user regarding the amount of recent system of usage in comparison with long-term trends in actual usage. Through this approach, it can be seen if the clinical use of the product with a patient is in line with what is prescribed clinically.

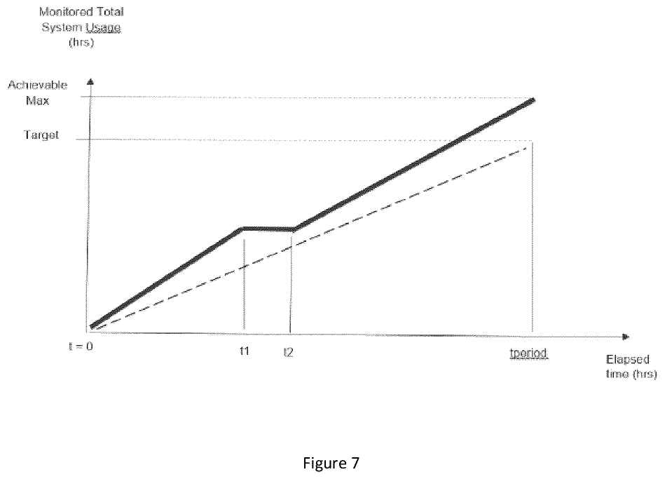

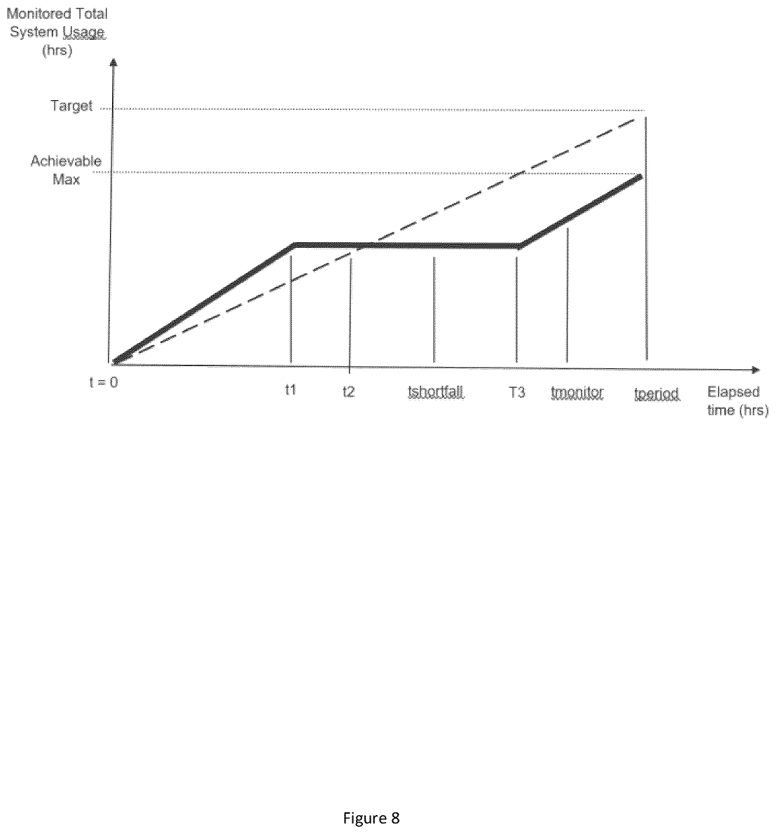

This functionality can be yet further extended to include an optional indication if the trend is likely to achieve a selectable usage threshold. An example of this involves comparing actual usage with that necessary over time to achieve a defined amount of usage associated with clinical policies or recommendations. This is described graphically in FIG. 7 & FIG. 8, where differing usage examples are shown.

FIG. 7 shows an example of the usage of a compression system over time from a starting point (t=0) to a future time (t_{period}), this timing is shown on the x-axis and could be a selectable period ranging from multiple hours to multiple days. During this period, it can be expected that a prescribed target usage of the compression system is required, as shown as the target usage on the y-axis. The target being in the form of a selectable value or proportion of the intended operation time (t_{period}), such as 75% of each 24 hours. The amount of actual usage is measured (denoted as Max Achieved in the figure) and accumulated and shown on the y-axis, this can be seen incrementing in value during the periods of usage (from t0 to t1) and (t2-t_{period}) with a first display type active, whilst not incrementing during the period of non-usage from t1 to t2. In this example, the compression system can monitor usage and due to the short non-usage time (t2-t1) it is able to calculate a prediction that it is possible to achieve its target usage (shown as Achievable Max>Target). As a result, no warning associated with lack of prophylaxis is provided to the user and a positive indication can instead be provided.

FIG. 8 shows an alternative example scenario of the usage of the same compression system compared to that of FIG. 7, with a longer period of non-usage of duration (T3-t1). As a result of this longer duration, it is no longer possible to achieve the target amount of prophylaxis in the time remaining under analysis (t_{period}). As a result of this reduced usage, a likely shortfall can be calculated and the control system identify that it is not possible to use the compression system sufficiently in the remaining time to achieve the target level (i.e. Target>Achievable Max). The compression system is able to perform a predictive analysis and hence detect this future situation earlier in the use/non-use of the system (shown as t=t_{shortfall}) and hence provide an early indication to the clinician/user. This indication can allow alternative steps to be taken in the use of the compression system and patient care to the clinical benefit of the patient. A further aspect is that the compression system can be configured to adapt its operation based on this identified condition so that the usage and/or performance is further increased over the remaining use of the product. This increase usage is achieved through promotion via modified displays on the user interface 117 and an increase in compression performance is possible through the automatic modification of compression operational parameters (e.g. pressure or cycle time).

It should be obvious to anyone skilled in the art that the invention as described through the example shown in FIG. 7 and FIG. 8 can beneficially highlight the lack of usage earlier in patient care. This can help avoid situations where insufficient prophylaxis is occurring and ongoing and hence help increase the usage of the compression system before the situation is beyond correction. The predictive and proactive approach of the monitored compression system allows for improved patient outcomes and therefore can present an improvement over the prior art. The combination of the monitoring of previous usage versus required target usage provides more information to clinicians that can help to improve the clinical outcome of the use of the compression system.

It should be evident to those skilled in the art that both of the example usage and non-usage periods demonstrated in FIG. 7 and FIG. 8 can be formed from many individual smaller periods. The compression system is able to process these multiple periods in the same manner through an aggregated analysis approach that operates on all the episodes of monitored usage. The algorithm is further able to provide a metric in the form of a score that can be recorded in the patient records based on the nature of the usage, together with the level and the type of prophylaxis that has been delivered.

Other aspects of performance monitoring included in the invention include the monitoring of the tightness of the fitted garment(s) on the patient limb or other anatomical area using the measurements taken by the compression system during the inflation and deflation of the compression garment(s). This forms part of the invention and provides a further means of assessing the effectiveness of the delivered prophylaxis and hence providing another aspect of usage and performance monitoring. This can be used to detect if individual compression garments are suitably fitted by the clinician or patent and provide control adjustments or indications to the user. This aspect of the invention allows the effectiveness of the compression to be identified in terms of a direct feedback measurement to be included in the algorithm described herein.

Instead of a simple compliance timer based on pump run time, as is well known in prior art, the monitoring system can also be used as a compliance timer that analyses and measures the summative effect of multiple individual episodes of use based on detecting limb presence in a compression garment and hence provide a measurement of the actual delivered prophylaxis to a limb. The approach does not rely on a simple totalization of operating times but instead seeks to identify, totalize and analyze individual aspects of each compression period of use with a patient and the periods and durations of non-use. This approach therefore offers a more useful clinical function than traditional compliance monitoring based on a simple overall totalized timing. The current invention provides monitoring and display of how long the prophylaxis has currently been in applied use with a patient limb and/or how long it has not been in applied use on a limb.

In an aspect, the pump monitors and displays the elapsed time since the garments were removed from the patient limb(s) or the garments(s) unplugged the garment to pump connector(s), both of which are possible without requiring access to the pump controls. It is this type of patient uncontrolled activity and behavior that the invention monitors and as this is believed to be a major cause of insufficient prophylaxis in clinical practice. A real life example of this situation is where a patient removes the garment(s) from their limbs or the pump connectors in order to visit a bathroom but then fails to reapply the garment(s) to the limb or the pump when they return. In this situation, the compression system changes from the first display state to show the second display state, which will display the elapsed time since the removal occurred. This aspect allows the clinician to readily ascertain the duration of this lack of use and the resulting lack of compliance to the prescribed care. The clinician can make an informed determination on the safe and appropriate intervention for the patient in terms of the reapplication of the garment(s) and resumption of prophylaxis.

FIG. 1, shows the general arrangement of a fluid pressure control system 100, i.e. a compression system, known in the prior art and able to embody the present invention. In one embodiment, the fluid pressure control system 100 is a gas pressure control system such as a pneumatic control system or is based on any type of suitable fluid for the application with either detachable or integrated inflatable/deflatable articles such as compression garments.

In an alternative embodiment, the control system and compression garment are physically integrated together where the control system is mounted on to the compression garment. This format of construction also allows the other aspects of the invention described herein, including the usage monitoring aspects and so this integrated construction format lies within the scope of the invention.

The fluid pressure control system 100 comprises an inflatable/deflatable article 120, a pump 110 and a controller (not shown in FIG. 1) operatively connected to the pump 110. The controller is operatively connected to the pump 110 for controlling said pump 110. The pump 110 may be a pneumatic pump. The controller monitors the usage of the pump under various situations including a first state of operation applying compression to a patients anatomy, a second state of operation applying compression but without a patients anatomy present and a third state of non-compression such as being inactive/idle or switched off. The time within each state being recorded and analyzed independently, a display being configured to visually differentiate between each state such that that the elapsed time in each individual state is provided.

In an embodiment, the pump 110 is arranged to control fluid flow to and from the inflatable/deflatable article 120. Accordingly, the pump 110 is arranged to inflate or deflate the inflatable/deflatable article 120. In an embodiment the control of the inflation characteristics are selected from a plurality of available characteristics (e.g. compression pressure, rise-time, cycle time etc) via the pump user interface. In a preferred embodiment, these inflation characteristics are selected by means of automatically detecting the type of garment connected hence reducing the need for user interaction in the selection and configuration of the appropriate characteristics for the connected garment type(s). In a preferred embodiment, the automatic detection of the garment type is through the use of an identification component located in the garment connector specific to each garment type. In a further embodiment the identification component located in the connector of each connected garment is used to select the monitoring means, algorithm and parameters for each individual connected garment type.

The fluid pressure control system 100 further comprises a coupling assembly 300 for fluidically connecting the inflatable/deflatable article 120 (e.g. compression garment) and the pump 110. The coupling assembly 300 comprises a connector 330 and a connecting member 310.

The connector 330 is connectable to the connecting member 310 to upon engagement form a fluid pathway through said connector 330 and the connecting member 310. Accordingly, the connector 330 may be connectable to the connecting member 310 to allow for fluid communication through the coupling assembly 300. In one embodiment the fluid flows through the identification component 390, the identification component being in the form of a ferrite or brass material, for example formed into a cylinder or core shape, and mounted within the garment connector 330 such that the identification component 390 substantially surrounds the fluid flow within the garment connector 330.

The compression system 100, shown in FIG. 1 in the form of an example format comprising a controller/pump 110, includes a microcontroller 210 located internally and providing software-based functions, a user interface 117 comprising visual and audible elements and at least one means of connecting 114 patient mountable compression elements to the system 100.

The compression elements shown in the example form of at least one compression garment 120 for mounting on a patients limb or anatomy, the compression garment being attachable 300 to the connecting means 114 via connector arrangements CA comprising separable physical connector parts 310, 330. The connector part on the garment 330 having a body 331 and including an identification component 390 such that the controller/pump can detect the type of connected garment from amongst a plurality of different types as well as a number of parameters associated with the monitoring of the connected garment type. The controller/pump 110 is configured to be able to monitor the use of the compression element/garment 120 when in use and when not in use by an individual patient and provide metrics and analysis of its status. Further, the system being able to monitor its degree of usage over an extended time across multiple patients to identify information and trends associated with its use, for example within a medical facility.

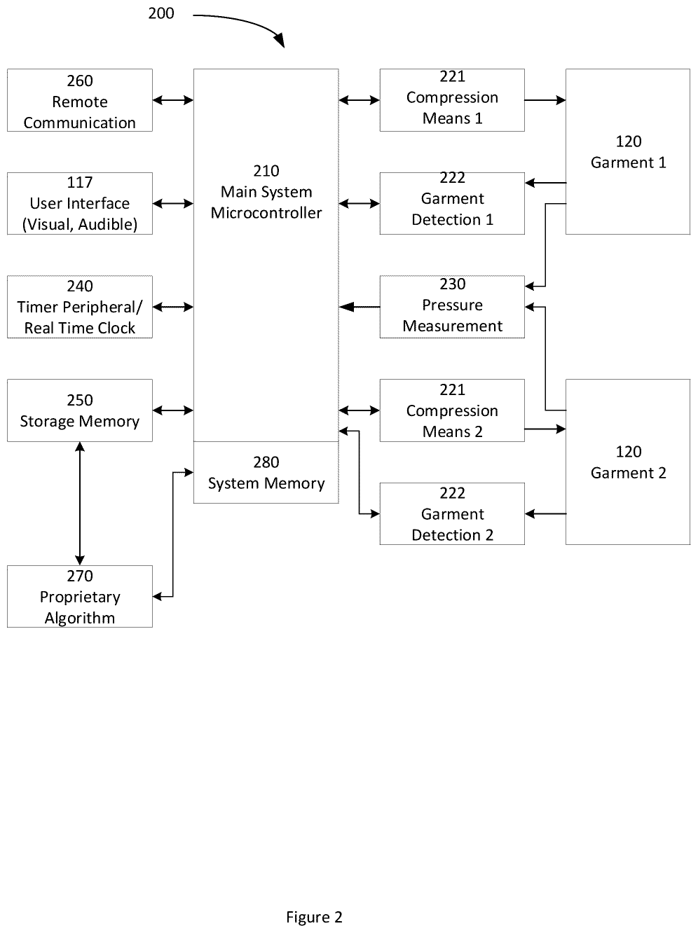

FIG. 2 shows a logical arrangement of elements of the control system 200 found in the compression system 100. With respect to FIG. 2, the arrangement of the control aspect is shown together with individual functional and logical elements. These elements are shown as individual elements for ease of explanation but it is within the scope of the invention that these can be physically combined in some embodiments. An aspect of the invention includes the provision of a proprietary algorithm intended to monitor the usage of the compression system over time and to estimate its effectiveness as a prophylactic or therapeutic medical device.

In an embodiment, at least one garment type is provided for attaching to an anatomical structure of a patients anatomy, for example on a limb such as foot, calf or thigh. The garment provides the compression into the patient anatomy either directly through its function (e.g. inflation) or by providing a mounting and location capability for an alternative compressive means such as an inflatable chamber or other device. Each connected garment sensed by a garment detection element 222 and this allows the main system controller 210 to identify and configure the required compression for each connected garment based on its detected type.

The compression provided by each connected garment 120 via a compression means 221, the compression means 221 providing and removing compression in the garment (for example using pneumatics with an air source and vent valve arrangement to inflate areas of the garment or by other means of direct compressive force to the patient anatomy). This compression means 221 can be shared across multiple garments or provided independently for each garment.

Feedback provided by the compression means 221 to the main system microcontroller 210 on the degree of engagement with the anatomy and the effect of the compression. As known, the compression pressure in an inflatable garment is readily monitored by the main system microcontroller 210 using a pressure measurement element 230 (for example a pressure transducer). This allows for the accurate and real time control and delivery of the compression to the garment 120 as well as detecting leak and error conditions. Additional feedback from the compression means 221 such as other non-pressure measurements can be used to ensure detect, correct and consistent delivery of the compression to the anatomical area.

The main system microcontroller 210 can provide visual feedback to the user on the status of the compression system 100 via a user interface 117 consisting of visual indications such as a LCD display, such as shown in FIG. 1 and in detail in FIG. 3a and/or with other LED based indicators located as part of the compression system. The user interface 117 can also provide audible feedback to the user using speakers, piezo sounders or other forms of audible devices. The user interface 117 can also provide for the control of the compression system 100 through the selection of system operation, operating modes, settings and other user-selectable features via the use of buttons, keypads and other forms of user inputs. FIG. 3a shows an example graphical user interface 117 of a compression system 100 known in the prior art in the form of a LCD panel with a full set of available graphic icons displayed. Only certain graphic icons being displayed at different times and in different operating modes. These provide indications of various functions and state of the operation of the compression system during different modes of operation and at different times.

One aspect of the operation and the invention is that the displayed pressure is displayed (for examples in convenient units such as mmHg), along with the detected garment being monitored for use. The displayed pressure being an indication of delivered pressure to the garment and a patient anatomy, the range of pressures being associated with the connected garment type as identified by the identification component located within the garment connector. An example embodiment involving the preferred target pressure to a first garment type intended for use on the leg, the target pressure being <65 mmHg and the duration of application and the actual pressure delivered being monitored by the control system and recorded. A further example embodiment involving the preferred target pressure to a second garment type intended for use on the foot being <140 mmHg and the duration of application and actual pressure delivered being monitored by the control system and recorded.

A timing element 240 is provided, for example in the form of a real time clock (RTC), that can be used to provide temporal-based information such as timing duration, clock and calendar information that can be used by the operating software stored in the system memory 280 and running on the software main system controller.

A storage memory element 250 is provided that allows the main system microcontroller 210 to store data and records associated with both periods of system usage (i.e. that intended and prescribed) as well as periods of non-usage (i.e. operation or operation contrary to that intended or prescribed) and includes timing information provided by the timing element 240. This allows a non-volatile storage of data associated with a typical period of patient care (e.g. lasting over hours and days) as well as data associated with the usage of the pump with multiple patients (e.g. over weeks and months). This data can be accessed and analyzed to provide information on the effectiveness of the system use by staff within a healthcare facility. An aspect of the invention is that the compression system memory 280 is suitably sized to be able to hold multiple activity records of use and non-use comprising a period of at least 3 months to allow for quarterly-based analysis and reporting. In a further embodiment the storage memory 250 is sized to store multiple activity records of use and non-use comprising a period of at least 6 months to allow for additional data archiving and retrieval benefits.

The main system microcontroller 210 is able to communicate this usage information from the storage memory 250 to external devices using remote communication element 260 (e.g. wirelessly using Bluetooth or Wi-Fi or using a wired connection such as USB).

Further referencing to FIG. 1, the fluid pressure control system 100 further comprises an indicating device 117, a user interface. The indicating device 117 is operatively connected to the controller. The indicating device 117 is configured to provide an indication to a user based on the identification of garment and/or an indication based on usage.

In an embodiment, the indicating device 117 is provided on the pump 110. Accordingly, the indicating device 117 is mounted to the casing of the pump 110. In one embodiment, the indicating device can also be mounted on the connecting member 310. Thus, the user is provided an indication of connection and usage while operating the connection to the pump 110.

In one embodiment, the indicating device 117 is mounted on the compression garment 120, thus the user is provided with an indication at the anatomical point of compression. The purpose being to show the usage signal and status at the point of physical attachment to the anatomical site and hence visually influence the removal process thereby promoting increased usage.

In one embodiment, the indicating device 117 is a display unit, such as an LCD-display. In one embodiment the LCD display uses graphical icons associated with the connected garment type as indicated by the identification component on each connected garment.

In one embodiment, the controller is provided on or within the pump 110. In another embodiment, the controller is provide on or within the garment 120.

As shown in FIG. 3a, the indication device 117 in the form of a graphical LCD display is shown with a complete range of icons associated with the various different functions associated with a compression system.

FIG. 3b highlights the specific icons from the panel shown in FIG. 3a used for the display of the usage state of the system when no garments are connected, i.e. prior to the use by a patient. This element represents the display when the product is not in prophylactic use. The display shows no connected garments are currently sensed as being connected to the pump.

The totalized previous usage of the system can be shown in the right hand lower corner 118 of the display in the form of a multi-digit digit elapsed time meter (e.g. shown as 888 hours) that can be used to indicate durations in hours and minutes. The display image also includes multiple graphic icons of sections of an example limb 117b for each compressive output of the compression system.

FIG. 3c shows an example display of the compression system when the system has detected the connection of two garments intended for connection to a patients legs, specifically on the calf and available for monitoring. A set of connected garment icons is visually indicated 117c and the usage information shown on the right hand lower corner 118 of the display in the form of a multi-digit elapsed time meter and is associated with the totalized and continued use of the compression system and optionally the connected garment type. FIG. 3c shows only those icons from FIG. 3a that are associated with the indication of a first display state 117a, 117c associated with usage monitoring with a patient. This comprises icons associated with an anatomical structure such as a limb with foot, leg and calf, various garment icons mounted on areas of the limb and a timing display 118 including a multi-digit seven segment numerical display. The nature of the garment icons displayed depends on those detected by the garment detection means. It is within the scope of the invention that different icons are used for different garment types. The timing information shown on the timing display is associated with the monitored use of the connected garments.

FIG. 3d shows an example second state display 117d of the compression system operating with the same connected calf garments as detailed in FIG. 3c but where the patient's limbs are not being present. The usage information shown on the right hand lower corner 118 of the display 117 is able to be in the form of a multi-digit elapsed time meter and the timing information is changed to be associated with the lack of use of these garments. This monitored timing information is shown in minutes initially, followed by hours and minutes dependent on the value of the monitored elapsed time.

This display condition is associated with the condition when the patient limbs have been sensed as not being present and the compression system is not delivering the prescribed therapy or prophylaxis. The timing information shown on the timing display is associated with the monitored lack use of the connected garments.

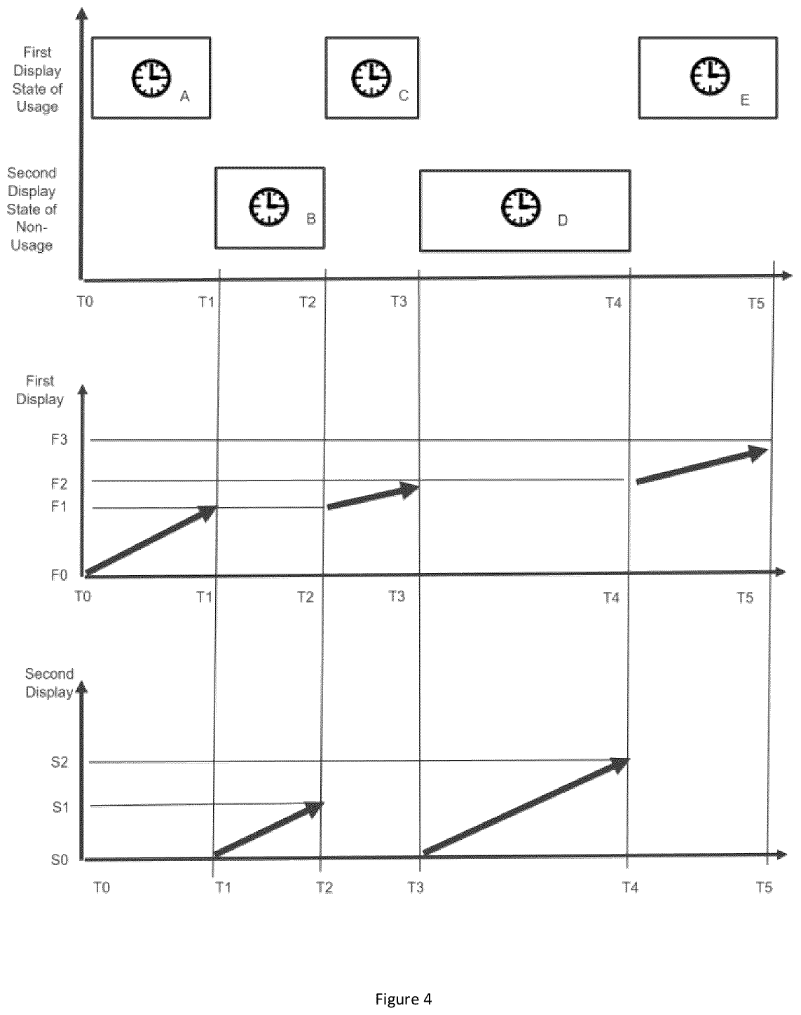

With respect to FIG. 4, this shows pictorially an example typical usage scenario of the compression system of FIG. 1 with a patient. The figure graphically shows the periods of use and non-use and the effect on the first display state and the second display state provided by the user display.

This usage scenario involves individual periods of usage A, C, E which are interleaved with periods of non-usage B, D. These periods can last for minutes or hours within the care of a typical patient and are typical of that experienced in a hospital environment. Examples of periods of usage A, C, E include operation of the compression system when the patient is located in a hospital bed, during surgery or sleeping and the compression system is specifically required and intended to be in use. Examples of typical non-usage periods include when the patient is attending a diagnostic procedure (e.g. X-ray) or having personal hygiene (bathing/toileting) when the compression system is specifically not intended to be in use.

A further and important consideration is that the periods of non-usage can occur during the time that the system is actually intended to be used. Examples of this scenario include to the physical removal of the garments from the patient's limb for an examination then the caregiver failing to replace them. In this scenario the patient is at risk as they are not receiving the prescribed therapy. This aspect is specifically monitored by the invention.

The timings are described accordingly. The first state of usage A lasts for a time from T0 to T1, the duration of which being T1-T0, the LCD display provides a first display state with a timing value increasing during T0 to T1.

This is followed by period of non-usage B, where the second display state is shown. This is followed by a period of usage C where the first display state is shown, a further period of non-usage D with the second display state visible and a final period of usage E with the first display being active.

The occurrence and duration of these individual periods of usage and non-usage is specific to each patient and so the resulting metrics provided by the usage monitoring and performance monitoring function of the compression systems can provide a means of quantifying this.

With reference to FIG. 4, an illustrative example is presented of the present invention of a compression system with two defined states of usage. The first state of usage being associated with the intended normal operation with a patient, the second state being associated with the operation of the system in an unintended mode resulting in lack of delivered prophylaxis. The system being able to monitor and interpret the results of these states across multiple individual episodes.

A number of discrete usage episodes are shown in a chronological sequence A, B, C, D, E where episodes A, C and E involve the correct operation of the system delivering compression therapy to a patient as intended and prescribed. Episodes B, D denote examples of usage where the compression is not delivered as intended or prescribed. Many factors can result in the situation shown in episodes B, D such as removal of compression garments from the limb of a patient or disconnection of the garments from the pump or detected fault conditions such as leaks.

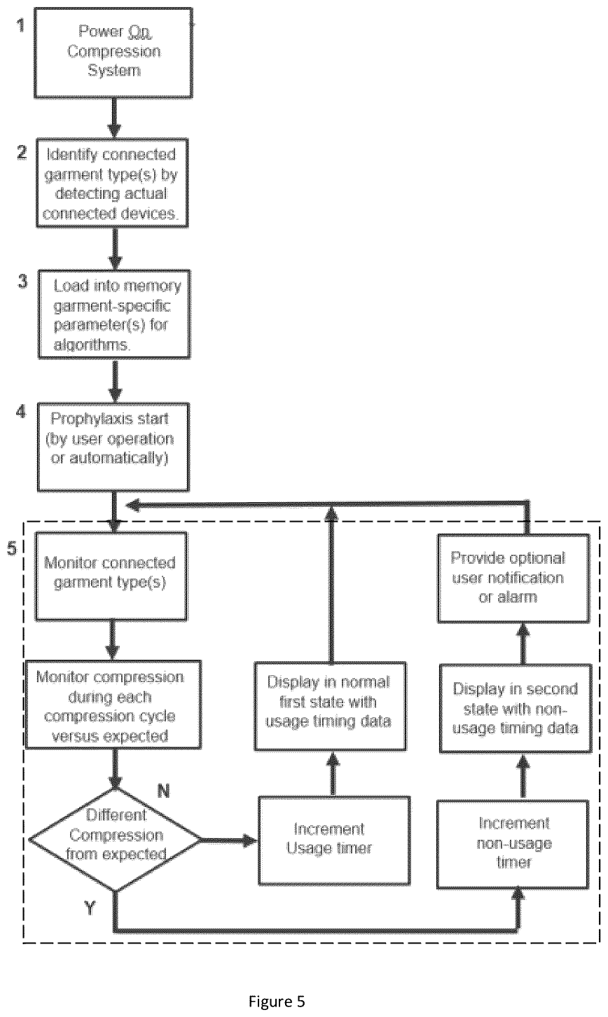

FIG. 5 shows a flowchart detailing an example embodiment of the invention that allows the determination of which display to provide on the compression system as a result of the monitoring system. With regard to FIG. 5, the system monitors each connected garment and controls its inflation in real time and is able to sense the presence of a limb within each compression garment during each inflation. This is achieved though analysis of multiple parameters and readings taken at different times during the inflation process. These readings include the pressure in the connected tubing, the pressure in the connected inflated garment, connected garment type and the compressor parameters required for each connected garment type as well as involving signals associated with the means of compression (such as amplitude of compressor drive waveform, frequency of applied drive waveform, current, voltage).

A measurement of prophylaxis delivery is created by combining the above parameters and is compared with expected readings for that specific connected garment type as well as that achieved on the previous inflation of that connected garment. The system looks for significant difference between these two analysis points and uses this difference to identify effectiveness of compression.

The compression system is able to change interchangeably between first and second display states based on the measurement of the presence of the limb in the garment. This change of display can occur with operation of only a single connected garment or with two connected garments. The change of display can occur if either of both out of two connected garments is detected as not being used with a patient's limb.

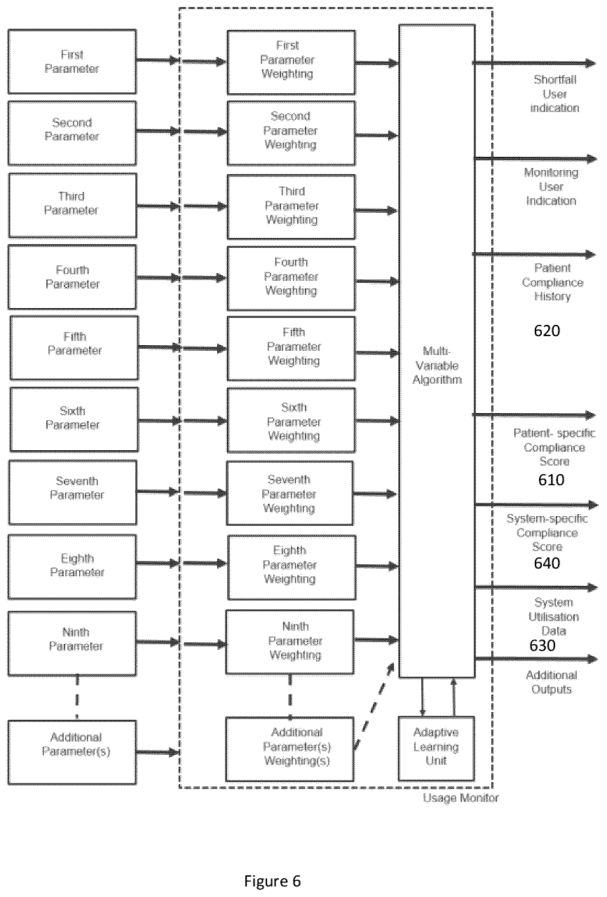

A further aspect involves the use of a proprietary algorithm shown diagrammatically in FIG. 6 via a generic algorithm structure that can be used to provide usage monitoring functions for the compression system. Each parameter is calculated by the compression system and provided with an individual parameter weighting and applied as an input to the algorithm.

It is within the scope of the invention that additional parameters can be added to the algorithm, and additional outputs can be generated by the algorithm.

Outputs of the algorithm are shown and are associated with the results of the interpretation of the individual patient use and non-use of the compression system. Some example outputs shown are described below.
A patient-specific compliance score 610 based on the monitored use with an individual patient, this could be in the form of a percentage or ratio of used time to non-used time as calculated over a defined time period.
A patient compliance history 620 comprising multiple individual measurements of usage over an extended time, such as a day-by-day score showing compliance with the required protocol.
System utilization data 630, indicating utilization of the compression system such as how much time the compression pump has been used over an extended period compared to its lack of use.
A system-specific compliance score 640 showing how the compression system has been used compared to a target degree of usage against a set threshold or target.

When the compression system senses that it is not delivering prophylaxis to a patient it can adapt its indication to the user. The user indication can involve LCD-based indications, LED based indications and audio indications based from the pump. Hence, the level of indication can be adapted as needed in a clinical setting. This can include a phased and progressive indication that is proportional or associated with the duration of the usage event. Hence, a low level of indication or priority can be provided to the user if the lack of usage has been of a short duration but this can be enhanced as the duration increases and the situation associated with lack of use becomes more significant.

In a preferred embodiment, as well as the first and second displays, a flashing green LED indication is provided at multiple locations on the compression system when a duration of 10 compression cycles have been sensed without correct prophylaxis being delivered. The use of multiple LED indicators on a compression system allows for multiple viewing directions ensuring that the state of the compression system and its usage can be readily determined by clinical staff from a variety of directions without having to approach or interact with the compression system.

This LED indication can be further increased in terms of its priority as the non-used time increases, for example though the use of a yellow LED alarm indication instead of a green LED. This change occurs if the number of cycles without the correct delivery of prophylaxis reaches to say 20 cycles.

It is also within the scope of the invention that the indications and timing data provided by the usage monitoring display on the compression system can be replicated at other locations physically away from the compression system.

The compression system can optionally include a means of communication of its status and data to a remote facility such as central monitoring equipment, a computer, tablet device, telephone or another device capable of displaying the status of at least one compression system or multiple compression systems. This communication means could utilize wireless technology (such as Bluetooth, ZigBee or Wi-Fi) and/or a dedicated wired connection to another device capable of displaying the information itself or providing onward communication such as a hospital bed or other medical device such as a support surface pump.

FIG. 7 shows the operation of the usage monitoring function of the compression system and an aspect where the system is being used effectively for an extended period, and the usage is above a set or calculated threshold or target. The detected usage is shown as an incrementing and monitored usage time (in hours) plotted against the y-axis with only a short period t1 to t2 when usage does not occur. The required target threshold (shown as the dashed line) is shown to be incrementing as time elapses.

Since the usage is more than the target, the use of the compression system can be considered sufficient and appropriate positive messaging and feedback is provided to the user. A score is calculated from the algorithm that can be recorded against the target indicating a satisfactory situation.

FIG. 8 shows the operation of the usage monitoring function of the compression system where the system is not being used as effectively as that shown in FIG. 7 such as due to a longer lack of use period t1 to T3. As a result the usage is below a set or calculated threshold or target. Since the usage is less than the target, the use of the compression system can be considered insufficient and appropriate negative messaging and feedback is provided to the user. A score is calculated from the algorithm that can be recorded against the target indicating an unsatisfactory situation.

A further aspect of the invention involves the analysis of the usage data of the compression system to determine whether it is above a calculated threshold associated with a selected compression regime. An example regime is the CHEST guideline published by the American College of Chest Surgeons which recommends mechanical prophylaxis is used for >18 hours in every 24 hours.

In an aspect, a control unit or a controller for controlling compression therapy to a patient's limb or anatomical structure using of at least one inflatable compression garment comprising at least one inflatable chamber. The control unit comprising means for detecting a type of connected compression garment using a measurement of an identification component located as part of a connector of the compression garment. The identification component being made of ferrite, steel or brass material.

The compression system comprising the following elements and aspects:
a connector/outlet port of the compression system in fluidic communication with at least one garment, the outlet port being able to detect which garment type is connected from a plurality of possible garment types,
an air pump for supplying and regulating air pressure to the compression garment based on the detected type, aspects of the delivered compression being dependent on the identification component sensed from the connected garment, at least one pressure sensor for measuring the pressure present in at least one of the inflatable chambers of the connected garment during the compression therapy and generating signals corresponding to the pressure in the chamber,
the pressure sensor providing signals during the application of compression comprising characteristics of the presence of the limb or anatomical structure within the at least one compression garment,
the pressure sensor denoting a change in the compression level of the compression garment as a result of the limb or anatomical structure being removed from the compression garment compared to the characteristic during the previous inflation,
a visual indication provided by the control unit to the user based on the monitored time when the limb or anatomical structure is present in the connected garment compared to the monitored time when the limb is detected as not being present in the connected garment.

In an aspect, a control unit for controlling compression therapy to a patient's limb or anatomical structure using of at least one inflatable compression garment is provided. The control unit is configured for correlating the connected compression garment type with measurements obtained from a pressure transducer connected to a pump outlet to form a detectable characteristic associated with the presence of a limb or anatomical structure surrounded by the at least one inflatable compression garment.

In an aspect, a control unit for controlling and monitoring the operation and delivery means of prophylaxis impulses to a limb or anatomical structure of a patient is provided. The control unit measuring a parameter forming a detectable characteristic of the presence of the limb or anatomical structure in contact with the compression garment

In an aspect, a control unit for controlling and monitoring the operation and delivery of compression therapy, using at least one inflatable garment, is provided. The control unit is in electronic communication with a separate patient-mounted remote sensor. The sensor measuring patient specific parameters and including at least one of the following:
the control unit performing an analysis of the signal(s) received from the sensor,
the control unit detecting the correlation between a change in the patient sensor with the operation of the control unit such that a change in the movement of blood in the anatomical structure is detected as a result of the operation of the control unit,
the control unit providing an indication to the user that communication is established with the remote sensor,
the control unit monitoring the patient usage of the garment over elapsed time based on the remote sensor signal.

In an aspect, the patient specific parameter is associated with the circulation status of the patient connected to the remote sensor.

In an aspect, the sensed parameter is associated with a measured change in the blood flow in the patient anatomical structure connected to the remote sensor.

In an aspect, the change in circulation of the patient anatomical structure is associated with the operation of the control unit.

In an aspect, the sensing occurs solely during the compression of the limb by the control system.

In an aspect, the detectable patient characteristics include at least two measurable parameters from the following list of parameters: connected garment type, detected garment tightness on the limb, measured volume of the garment, temperature of the limb, detectable arterial pulse in the limb, pressure response to an applied pressure impulse into the garment, rate of rise of chamber pressure during inflation, variation in pressure during the compression, rate of fall of chamber pressure during deflation, compressor setting, a communicated patient parameter from external equipment.

In an aspect, the presence of a limb in the garment is defined and confirmed by the combination of at least two different measured parameters.

In an aspect, a visible indication is shown on the control unit of the presence of the patient limb in at least one of the connected garments.

In an aspect, a visible indication is shown on the control unit of the removal or absence of a detected limb in at least one of the connected garments.

In an aspect, the visible indication is in the form of a graphical limb image.

In an aspect, the indication of the presence of a limb in the garment is also provided remotely to separate monitoring equipment located away from the control unit.

In an aspect, the garment inflatable chamber is located most distally amongst a plurality of inflatable chambers in the garment.

In an aspect, the value of the measured parameter varies between different garment types.

In an aspect, the value of a measured parameter is based on a garment presence on a limb.

In an aspect, the value of the measured parameter is based on a garment effect on a limb being compressed.

In an aspect, the value of the measured parameter is correlated with the sensed connected garment type to provide confirmation of the presence of a limb in the specific connected garment type.

In an aspect, the measured air temperature in the connected garment is elevated if a limb is present in the garment compared to the air temperature in a garment with no limb being present.

In an aspect, the applied therapy parameters are varied in response to the measured parameter.

In an aspect, pressure level applied to the garment is varied as a result of the measured parameter.

In an aspect, the cycle time of the garment is varied as a result of the measured parameter.

In an aspect, the inflated time of the garment is varied as a result of the measured parameter.

In an aspect, the operation in conjunction with a limb present in at least one of the garments is recorded in a memory, such as a digital memory.

In an aspect, the duration of operation is recorded in a digital memory

In an aspect, the time and date of limb detection is stored in a memory.

In an aspect, the duration of the operation in conjunction with a limb present in at least one of the garments is recorded.

In an aspect, the time and date of the lack of limb detection is stored in a memory.

In an aspect, the control unit is capable of recording the duration of time when it is not in use.

In an aspect, the control unit is capable of recording the time and date when the control unit is not in use with a patient.

In an aspect, data is paired to construct start/stop patient episodes for usage detection, monitoring and analysis.

In an aspect, the control unit combines the start and stop of limb detection via data pairing to form a record of a patient use episode that corresponds to the use of the control unit.

In an aspect, where there is a gap between patient usage episodes, a control unit combines the stop and start of limb detection via data pairing to form a non-use episode that corresponds to when there is no use of the control unit by the patient.

In an aspect, the paired episode details are recorded in a memory, such as a digital memory.

In an aspect, the contents of the digital memory associated with each episode of limb detection in the at least one garment can be communicated to external equipment.

In an aspect, the contents of the digital memory associated with each episode of limb detection in the at least one garment are displayed on the control unit.

In an aspect, where indication of sufficient/insufficient prophylaxis is put against a defined target threshold, a control unit for providing monitoring of the cumulative duration of the detected limb detection episodes is provided.

In an aspect, an indication is provided to the user when the cumulative duration of limb detection episodes exceeds a target threshold over a defined period of elapsed time.

In an aspect, an indication is provided to the user when the cumulative duration of a limb detection episodes is below a target threshold over a defined period of elapsed time.

In an aspect, specific progress indications is provided, a control unit is provided wherein the monitored duration is displayed as a percentage of the target threshold.

In an aspect, the duration is shown as a visual part of a graphic icon representing the target threshold.

In an aspect, where usage is changing, a tendency indicator metric is calculated showing either an improvement or deteriorating condition in terms of usage. A control unit is provided, where a tendency indication is provided to the user when the monitored usage is changing over time. The monitored usage being comparable to a threshold, the threshold being defined by the identification component associated with the garment detector.

In an aspect, an indication is provided to the user when the monitored usage is decreasing over time.

In an aspect, an indication is provided to the user when the monitored usage is increasing over time.

In an aspect, a target threshold is provided, wherein the target threshold is user adjustable in the control unit.

In an aspect, the target threshold is associated with a defined elapsed time.

In an aspect, the defined period of elapsed time is user adjustable in the control unit.

In an aspect, a target threshold is provided is related to each connected garment type. The target threshold is associated with the specific garment type attached to the control unit, the target threshold being stored in the control unit.

In an aspect, it may be necessary or beneficial to set different targets based on the need of the patient. A control unit is provided, wherein the target threshold stored in the control unit is updated by the measurement of a parameter from the connected garment type.

In an aspect, the required target threshold is user adjustable based on the clinical condition of the specific patient.

In an aspect, the required target threshold is user selectable between a number of preset values.

In an aspect, the required target threshold is user selectable based on the clinical policy of the individual healthcare facility using the control unit.

In an aspect, the control unit monitors to the CHEST guideline clinical requirements, where the required target threshold is 18 hours of usage out of every 24 elapsed hours.

In an aspect, an adaptive threshold based on detected usage is added-if the system monitored usage is less than the required threshold, the threshold is adjusted and is increased for the next usage time period. A control unit is provided, where the target threshold automatically varies based on the monitored usage over the monitored time period.

In an aspect, a risk value is calculated by using a known clinical assessment method e.g. Caprini, Padua, Rogers, Potter, This risk value is used as basis for selecting a threshold. A control unit is provided, where the user can adjust the usage threshold based on a clinically accepted risk-scoring system.

In an aspect, the individual target thresholds equate to a risk scoring system with multiple levels of selectable thresholds.

In an aspect, a communication link to patient records is added-both for reporting the adherence to the threshold and also allowing the setting the threshold.

A control unit is provided, wherein the data regarding the adherence to target/threshold and individual patient use episodes are electronically transmitted and stored in a patient's electronic medical record. Examples of electronic communication includes wireless means such as Bluetooth, WiFi, Zigbee and the use of wired connection such as USB.

In an aspect, the control unit is capable of receiving via electronic transmission a target threshold value from a patient electronic medical record. The compression system operation being capable of being adapted to indicate or otherwise recommend a prescribed usage target based on a clinical prescription, this target being translated into a usage threshold associated with each garment type as identified by garment identification components.

In an aspect, the compression system includes the ability to adaptively modify or otherwise change the target threshold based on temporal data such as time or elapsed time (e.g. employing different thresholds based on the time of day to compensate for significant use differences such as between nighttime sleeping and daytime clinical activities). A control unit is provided, wherein the required threshold is automatically varied dependent on the time of day to provide a varying threshold.

A further aspect involves the use of different thresholds dependent on the monitored duration of usage. For example, having higher target thresholds at the start of patient usage such as prior to surgery or during initial hospitalization and then progressively a lower as patient recovery and mobility occurs.

In an aspect, a fluid pressure control system or a compression system is provided. The system comprises a pump, at least one inflatable/deflatable article such as a compression garment, an indicating device such as a user display, a monitoring system or a control unit for detecting the usage of the at least one compression garment. The user display comprises a first display indicator associated with the usage of the compression garment in a first state, e.g. a first prophylaxis state or a first operational state, of operation of the compression system. The user display comprises a second display indicator associated with the usage of the garment in a second state, e.g. a second non-prophylaxis state or a second operational state, of operation of the compression system.

In an aspect, the first state of operation is associated with the presence of a patient's limb within the garment and the second state of operation is associated with the lack of presence of a patient's limb within the compression garment.

In an aspect, the monitoring system being configured to monitor time when the compression garment is in each state of operation.

In an aspect, the first state of operation is a delivered prophylaxis state and the second state of operation is a non-delivered prophylaxis state.

In an aspect, the monitoring system being configured to monitor the delivered effectiveness of the prophylaxis to the compression garments.

In an aspect, the connected garment and presence of a patient limb is monitored by the monitoring system over time to provide a metric of system usage.

In an aspect, the metric formed from usage time over the monitored time is compared with a target usage threshold.

In an aspect, the compression system includes a remotely located patient sensor, the sensor connected to a patient anatomical structure, the sensor in electronic communication with a control unit for the control and monitoring of compression to the patient.

In an aspect, the compression system includes and provides the aforementioned means and a method for detecting the usage of the at least one compression garment of a compression system. The compression system comprises a user display. The user display comprises a first display indicator associated with the usage of the compression garment in a first state of operation of the compression system, and a second display indicator associated with the usage of the garment in a second state of operation of the compression system.

In an aspect, the compression system has a first state of operation is associated with the presence of a patient's limb within a compression garment and the second state of operation is associated with the lack of presence of a patient's limb within the compression garment.

In an associated aspect, the method comprising monitoring time when the compression garment is in each state of operation

In an aspect, the first state of operation is a prophylaxis state and the second state of operation is a non-prophylaxis state.

In an aspect, the method comprising monitoring delivered effectiveness of the prophylaxis.

In an aspect, the method comprising monitoring the connected garment and the presence of a patient limb by the monitoring system over time to provide a metric of system usage.

In an aspect, the method comprising comparing the usage state over the monitored time with a target usage threshold.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

The foregoing has described the principles, preferred embodiments and modes of operation of the present invention. However, the invention should be regarded as illustrative rather than restrictive, and not as being limited to the particular embodiments discussed above. The different features of the various embodiments of the invention can be combined in other combinations than those explicitly described. It should therefore be appreciated that variations may be made in those embodiments by those skilled in the art without departing from the scope of the present invention as defined by the following claims.

1. A control unit for analyzing usage of compression therapy to a patient's limb or anatomical structure by using at least one inflatable compression garment comprising at least one inflatable chamber, the control unit comprising:
an identification component for detecting a type of at least one compression garment, used during the compression therapy, and located within a connector of the at least one compression garment;
an air pump for regulating air pressure to the at least one compression garment based on the detected type; and
at least one pressure sensor for measuring the pressure present in at least one of the inflatable chambers of the connected at least one garment during the compression therapy and generating signals corresponding to the pressure in the chamber;
wherein the pressure sensor is providing signals during the application of compression which signals comprises characteristics of the presence of the limb or anatomical structure within the at least one compression garment,
wherein the pressure sensor denoting a change in the compression level of the at least one compression garment as a result of the limb or anatomical structure being removed from the at least one compression garment compared to the characteristic of the signals during the previous compression,
wherein a visual indication is provided by the system to the user based on monitored time when the limb or anatomical structure is present in the at least one connected garment, and
wherein a visual indication is provided by the system to the user based on monitored time when the limb or anatomical structure is detected as not being present in the at least one connected garment.

2. The control unit according to claim 1, wherein the control unit is configured for correlating the connected compression garment type with measurements obtained from a pressure transducer connected to a pump outlet to form a detectable characteristic associated with the presence of a limb or anatomical structure surrounded by the at least one inflatable compression garment.

3. The control unit according to claim 1, wherein the control unit is configured for:
monitoring the operation and delivery means of prophylaxis impulses to a limb or anatomical structure of a patient, and
measuring a parameter forming a detectable characteristic of the presence of the limb or anatomical structure in contact with the compression garment.

4. The control unit according to claim 1, wherein the control unit is in electronic communication with a separate patient-mounted remote sensor, wherein the sensor is configured for measuring patient specific parameters and including at least one of the following:
the control unit performing an analysis of the signal(s) received from the sensor,
the control unit detecting the correlation between a change in the patient sensor with the operation of the control unit such that a change in the movement of blood in the anatomical structure is detected as a result of the operation of the control unit,
the control unit providing an indication to the user that communication is established with the remote sensor, and
the control unit monitoring the patient usage of the garment over elapsed time based on the remote sensor signal.

5. The control unit according to claim 1, wherein the identification component being made of ferrite, steel or brass material.

6. A compression system for analyzing usage of compression therapy by using at least one compression garment, comprising a user interface, wherein the user interface comprises:
a first display indicator associated with the usage of the at least one compression garment in a first state of operation of the compression system, and
a second display indicator associated with the usage of the at least one compression garment in a second state of operation of the compression system.

7. The system according to claim 6, wherein the system comprises a control unit configured for monitoring the operation and delivery means of prophylaxis impulses to a limb or anatomical structure of a patient and measuring a compression parameter forming a detectable characteristic of the presence of the limb or anatomical structure in contact with the compression garment.

8. The compression system according to claim 7, wherein the compression parameters are capable of adaptation and modification based on the degree of previous usage based on records held in the memory storage device.

9. The compression system according to claim 8, wherein the compression parameters that are capable of being modified are based on a sensed identification component in a garment connector.

10. The compression system according to claim 8, wherein a compression pressure is adjusted based on the analysis of records of previous usage of the compression system stored in a storage memory of the compression system.

11. The compression system according to claim 8, where the cycle time of the compression system is adjusted based on the analysis of records of previous usage of the compression system stored in a storage memory of the compression system.

12. The compression system according to claim 1, wherein the detection of the presence of a limb in the at least one compression garment only occurs during the inflation of the at least one compression garment.

13. The compression system according to claim 1, wherein the sensing of the presence of the limb is based on at least one parameter from the list of pressure transducer, compressor drive parameters, garment connector type, identification component type or analysis of records of the previous use of the compression system stored in a storage memory of the compression system.

14. A compression system comprising:
a connector of the compression system in fluidic communication with at least one compression garment, the connector being able to detect which garment type is connected from a plurality of possible garment types;
an air pump for supplying and regulating air pressure to the at least one compression garment based on the detected type;
at least one pressure sensor for measuring the pressure present in at least one inflatable chamber of the at least one connected compression garment during the compression therapy and generating signals corresponding to the pressure in the at least one inflatable chamber;
the pressure sensor providing signals during the application of compression comprising characteristics of the presence of the limb or anatomical structure within the at least one compression garment;
the pressure sensor denoting a change in the compression level of the at least one compression garment as a result of the limb or anatomical structure being removed from the at least one compression garment compared to the characteristic during the previous inflation; and
a visual indication provided by a control unit of the compression system to the user based on monitored time when the limb or anatomical structure is present in the at least one connected garment compared to monitored time when the limb is detected as not being present in the at least one connected garment.

15. A method for analyzing usage of compression therapy by using at least one compression garment of a compression system comprising a user interface, wherein the user interface comprising:
a first display indicator associated with the usage of the compression garment in a first state of operation of the compression system; and
a second display indicator associated with the usage of the garment in a second state of operation of the compression system, wherein the method comprising monitoring time when the compression garment is in each state of operation.

16. The method according to claim 15, comprising monitoring delivered effectiveness of the prophylaxis.

17. The method according to claim 15, comprising monitoring the at least one connected compression garment and the presence of a patient limb by a monitoring system of the compression system over time to provide a metric of system usage.

18. The method according to claim 15, comprising comparing the usage state over the monitored time with a target usage threshold.
